(19) United States
(12) Patent Application Publication
(10) Pub. No.: US 2022/0017858 A1
(43) Pub. Date: Jan. 20, 2022

(54) SYSTEMS, DEVICES AND METHODS FOR IDENTIFICATION, SELECTIVE ABLATION, AND SELECTION AND COLLECTION OF SINGLE CELLS

(71) Applicant: IsoPlexis Corporation, Branford, CT (US)

(72) Inventors: Rui ZHENG, Branford, CT (US); Sean MACKAY, New Haven, CT (US)

(21) Appl. No.: 17/311,011

(22) PCT Filed: Dec. 4, 2019
(86) PCT No.: PCT/US2019/064535

§ 371 Date: Jun. 4, 2021

Related U.S. Application Data
(60) Provisional application No. 62/775,073, filed on Dec. 4, 2018.

(51) Int. Cl. C12N 5/00; C12M 1/00; G01N 33/543; G01N 33/569; G01N 21/64

(52) U.S. Cl. CPC C12N 5/0087; C12M 47/04; G01N 33/54386; G01N 21/6452; G01N 33/56972; G01N 33/56966; G01N 21/6458; C12M 47/06

(57) ABSTRACT

Embodiments of the present disclosure are directed to systems, devices, and methods for the selective collection of cells from a heterogeneous cell population, including highly multiplexed detection of secreted and intracellular macromolecules and the targeted laser-assisted ablation of cells identified to be positive or negative for a given biomarker or phenotype. The resulting non-ablated cells can be collected individually or pooled to form a homogenous cell population for further processing including safe and efficacious cellular therapies.

### CROSS-REFERENCE TO RELATED APPLICATION

This application claims priority to U.S. Provisional Application No. 62/775,073 filed Dec. 4, 2018, entitled “COMPOSITIONS AND METHODS FOR IDENTIFICATION, SELECTIVE ABLATION AND COLLECTION OF SINGLE CELLS,” the disclosure of which is incorporated by reference herein.

### FIELD OF THE DISCLOSURE

The disclosure is directed to systems, devices and methods for recovery and retrieval of biological material, and more specifically, to methods selectively collecting (and in some embodiments, eliminating) cells in view of identified secreted and intracellular components thereof via highly multiplexed secreted protein (for example) from single cells in single wells/chambers, and fiducial combinations to make such identification. In some embodiments, such systems, devices, and methods can also be used with collection and/or elimination of a homogeneous population of cells.

### BACKGROUND

In the art, analyzing immune cells typically comprises analyzing secreted proteins and in particular cytokines, which are key mediators of intercellular communication within the immune system. Homeostatic immune response requires tightly regulated cytokine synthesis and secretion. Many analytical technologies have been developed to analyze protein secretion during the immune response, but the methods used are generally restricted to measuring the average secretions for an entire cellular population. Such analyses, while helpful in understanding disease pathogenesis and the immune process, are insufficient to characterize cytokine activity for individual subsets of cells within a population. Recent investigations using single-cell analysis have shown that immune cells display highly heterogeneous cytokine profile even in cells with similar phenotypes further demonstrating a significant limitation of focusing only on cellular response at the bulk population level. These heterogeneous subsets of cells within the population may dictate a complex signaling interplay between cells that represent important checks and balances for disease immunotherapy evaluation. This is particularly notable when a cellular population's response can be determined by the cell-cell interactions in a rare subset of cells. As result, understanding these interactions is crucial to developing more effective therapeutic treatments in the future.

Accordingly, challenges remain to defining consistent and high functional quality “drug” in cell-based cancer immunotherapy. Despite the demonstrated benefit of emerging CAR-T cancer immunotherapeutics, two major concerns remain: (1) manufacturing the cell therapies consistently, and (2) managing the immuno-toxicity, such as cytokine release syndrome, that could be potentially life threatening. In cell-based therapies such as Chimeric Antigen Receptor T cell (CAR-T) therapy, in which the living cells are the “drug”, cellular manufacturing is still relatively new, and each patient batch generated may differ substantially even if a standardized operation procedure (SOP) exists to ensure consistency in manufacturing. Giving clinicians and biotech companies an effective cellular function monitoring tool could change clinical paradigms by allowing them to remove or modify the inconsistent or unsafe cell therapies prior to injection, significantly reducing risk to the patient, and improving odds of therapeutic success.

To evaluate engineered T cells for an immunotherapy or to evaluate endogenous T cells reactivated to battle cancer or infection, a T cell's functional status is largely determined by a spectrum of secreted effector function proteins (e.g., cytokines). In a protective immune response, the ‘quality’ of an immune cell correlates to the extent of polyfunctionality (the ability of a T cell to co-secrete multiple effector proteins). While these anti-tumor cytolytic, chemoattractive cytokines produce an effective response, these poly-functional cell subsets must not also secrete immuno-toxic inflammatory or regulatory cytokines (up to 15) prevalent in NK or CAR-T cells. To detect consistent performance of this “quality” effective and safe response of CAR single-cell subsets, a need exists to conduct highly multiplexed measurement of immune effector proteins in single T cells.

Single parameter ELISpot assay remains the state-of-the-art for T cell activation assay, but does not measure polyfunctionality. Averaging bead based multiplexing platform (Luminex based) measure many cytokines, yet not at the required single-cell level. Fluorescence-based multicolor flow cytometry is a powerful single cell analysis tool and has been used to detect cytokines via retaining and staining proteins within the cytoplasm by blocking vesicle transport. The number of proteins that can be simultaneously measured is limited by fluorescence spectral overlap with ICS (intracellular cytokine staining).

Time-of-flight mass spectrometer-coupled flow cytometry (CyTOF) has been used recently by CAR-T companies, though not as regularly in trials. Similar to fluorescence flow cytometry, it does not measure true secretion and so far the number of cytokines co-measured in single cells by CyTOF is limited (e.g., 11) due in part to the high background of ICS. Other single cell technologies being developed in research laboratories (e.g., microengraving) provides advantages in sensitivity and assay speed but still limited in the multiplexing capability (typically <5).

Adoptive cell therapies and other immune mediated therapies pose risks. Cytokine release syndrome (CRS) is a non-antigen specific, life-threatening toxicity that results from the over-activation of the immune system due to immune therapies, such as CART-cell therapy. Although CAR T-cells are potent on-target killers, they activate the immune system far above naturally occurring levels and due to the nature of their design, have a large degree of “on target, off tumor” toxicity. Based on the level of mortalities in recent clinical trials, it has become apparent that the cytokine profiles of individual CAR-T cells must be known before introduction to the patient. The systems and methods of the disclosure determine an abundance of up to 42 cytokines, per single cell, falling into the following groups: effector, stimulatory, inflammatory, and regulatory. This information allows the user to identify any potentially toxic subsets of cells (pro-inflammatory or regulatory) within a population that would have been missed by conventional means, providing a safer and more effective means of monitoring immunotherapies prior to patient introduction.

Thus, there has been long-felt but unmet need in the art for a system, device and/or method of identifying, sorting, and collecting single cells or homogenous populations of cells from a heterogeneous sample of cells. The present disclosure includes embodiments for systems, devices, and methods to solve such long-felt but unmet needs.

### SUMMARY OF AT LEAST SOME OF THE EMBODIMENTS

Embodiments of the present disclosure provide systems, devices and methods for selectively collecting/sorting (and in some embodiments, eliminating) cells in view of identified secreted and intracellular components thereof. In particular, it is an object of at least some embodiments of the present disclosure to provide controlled identification and retrieval/extraction for proteomic expression from defined cells. This allows, in some embodiments, to collect one or more cells for its specific secreted proteomics information, or to identify one or more cells based upon its intracellular components (secreted proteins from the cell, or intracellular components can be used interchangeably throughout). Moreover, in some embodiments, selective laser ablation can be used for the purpose of retention and retrieval/collection of one or more cells for downstream analysis. Thus, in some embodiments, the systems, devices and methods are presented which yield a cell sorting system by, for example, a cell (or cells) true multipoint secreted proteomic profile (which can be used to deliver/collect a specific desired cell).

Accordingly, in some embodiments, a selective cell collection and/or sorting method for at least one of selectively collecting and sorting cells is provided, and includes loading or otherwise placing into each of a plurality of isolated chambers of a substrate, a cell and a volume of fluid. The substrate including a first surface that is releasably coupled to a transparent cover having a second surface, forming an assembly, the second surface having a plurality of capture agents, and the volume of fluid is in fluid communication with the second surface. The method also includes maintaining each cell under one or more conditions sufficient to permit the production of one or more cellular components by each cell, and the one or more cellular components configured to bind with at least one of the capture agents of the surface so as to form at least one capture agent cellular component complex. The method yet further includes, for each of the cells, detecting the at least one capture agent cellular component complex, and identifying at least one cell for at least one of collection and removal. The method may yet further include collecting the at least one cell.

The above noted embodiment may further include ablating a cell identified for removal, where ablating can comprise contacting a respective isolated chamber comprising the cell for removal with a laser. In addition, in some embodiments, the laser is configured to lyse the cell.

Such embodiments may include one and/or another of the following additional steps, features, associated structure, functionality and/or clarification (and in some embodiments, a plurality thereof, and in still yet further embodiments, a majority or all thereof), yielding yet further embodiments of the current disclosure:
each cell is selected from a plurality of cells;
the chambers may be arranged in rows and/or columns;
a plurality of cells comprises a heterogeneous population of cells, and the heterogeneous cell population can be a functionally heterogeneous cell population, comprising:
   at least two cells that produce a secretome in response to a stimulus, and a first cell of the at least two cells produces a first secretome, a second cell of the at least two cells produces a second secretome, and the first secretome and the second secretome are not identical;
   one or more immune cells which can comprise a T-lymphocyte, a B-lymphocyte, a natural killer (NK) cell, a macrophage, a neutrophil, a mast cell, an eosinophil, or a basophil, and the T-lymphocyte can comprise a native T-lymphocyte, an activated T-lymphocyte, an effector T-lymphocyte, a helper T-lymphocyte, a cytotoxic T-lymphocyte, a gamma-delta T-lymphocyte, a regulatory T-lymphocyte, a memory T-lymphocyte, or a memory stem T-lymphocyte,
      where the T-lymphocyte can express, for example, a non-naturally occurring antigen receptor, or a Chimeric Antigen Receptor (CAR);
      where the B-lymphocyte can comprise a plasmablast, a plasma cell, a memory B-lymphocyte, a regulatory B cell, a follicular B cell, or a marginal zone B cell;
   one or more neuronal cells, which can comprise a neuron, a glial cell, an astrocyte, a satellite cell, or an enteric glial cell;
   the functionally heterogeneous cell population comprises one or more endocrine cells, which can be isolated or derived from a pineal gland, a pituitary gland, a pancreas, an ovary, a testicle, a thyroid gland, a parathyroid gland, a hypothalamus, or an adrenal gland;
   one or more exocrine cells, which can be isolated or derived from a salivary gland, a sweat gland or a component of the gastrointestinal tract, where the component of the gastrointestinal tract can be a mouth, a stomach, a small intestine, and a large intestine;
the one or more conditions can be, for example, contacting each cell with a stimulus, where the stimulus:
   may be selected from the group consisting of: an ion, a small molecule, a nucleic acid sequence, a peptide, a polypeptide, a protein, a ligand, a receptor, an antigen, a cell or organelle membrane patch, a cell or organelle membrane, a cell, or any combination thereof;
   can be naturally-occurring or not naturally occurring;
   can be operably-linked to an interior surface of each respective chamber;
   the volume of fluid in the chamber can correspond/be the stimulus;
   recapitulates the effect of the subject cell contacting a target cell, where the target cell:
      can be a deleterious cell, and can be selected from the group consisting of: a proliferating cell, a cancer cell, an infected cell, a foreign cell, or an immune cell, where the foreign cell is a bacteria, a yeast, or a microbe,
      is a healthy cell, which can be a B-lymphocyte;
the one or more conditions can comprise maintaining each of the at least two cells in a cell media that maintains the viability of each cell;
the volume of fluid in each of the at least two isolated chambers includes a cell media that maintains the viability of each cell;
the capture agents can be arranged on the surface in a repeated pattern,
the substrate and the surface can be releasably coupled such that at least one repeat of the repeated pattern of capture agents is enclosed in each chamber of the plurality of chambers;
the one or more cellular components can comprise a secretome, and the secretome can comprise one or more distinct peptides, polypeptides, or proteins that indicates diminished or decreasing cell function or cell viability;
the one or more cellular components comprise a secretome, where the secretome can comprise one or more distinct peptides, polypeptides, or proteins that indicates:
   augmented or increasing inflammation; and
   increased cell activity or cellular stimulation;
the identifying step can comprise determining a Polyfunctional Strength Index (PSI) for each of the at least two cells, where:
   the PSI can be the product of a percentage of polyfunctional subject cells within the heterogeneous cell population and an average signal intensity of two or more cytokines, and the polyfunctional subject cells, at a single cell level, can secrete at least two cytokines (which can be the same cytokines, or different); and
   an increase in the PSI indicates an increase in the potency of the polyfunctional subject cells;
during the ablation step, the laser does not directly contact the cell identified for removal;
the laser is selected from the group consisting of: a diode, helium-neon, argon, xenon, nitrogen, carbon dioxide, carbon monoxide, hydrogen fluoride, deuterium fluoride, helium-cadmium, helium-mercury, helium-selenium, helium-silver, or neon-copper laser;
the collection step can comprise: flowing a collection fluid across the isolated chamber comprising the cell identified for collection to produce a composition comprising the cell, and/or capturing the at least one cell identified for collection using a pipette or a nanopipette;
   the composition can comprise the captured cell.
   the composition can be purified to remove one or more of a stimulus, a reagent, a cell media, one or more cellular components, one or more components of a secretome, a secreted protein, an intracellular component, cell debris or any combination thereof;
   the composition can include a media to maintain viability or polyfunctionality of the cell;
contacting the collected cell with an expansion composition;
analyzing the collected cell or a component thereof, where analyzing can comprise one or more of DNA sequencing, RNA sequencing, genomics analysis, and proteomics analysis;
detecting can comprise at least one of exposing the cover of the assembly to a light source configured to fluoresce formed complexes, and imaging the cover for fluorescing complexes;
imaging the cover can include taking a plurality of images of overlapping portions of both the first and second surfaces;
the second surface can be configured with a plurality of fiducial lines and the substrate is configured with a plurality of organized fiducial marks;
identifying can comprise using the fluorescence and lines and marks of the images to locate one or more of the chambers to which the fluorescence corresponds;
images can be assembled together (either initially, or subsequent to, e.g., processing);
fiducial lines can be arranged on the second surface in a repeated, pre-defined pattern;
   each fiducial line can include a unique color;
and
fiducial marks can be arranged on the first surface in-between columns of the chambers;
   the marks can be configured with two or more different length of with equal spacing between; and
   chambers in between the spacing can be labeled.

In some embodiments, a selective cell collection system is provided and includes a dock suitable, which in some embodiments comprises a clamp, for receiving a device comprising a substrate and a transparent cover including a second surface. The substrate includes a plurality of isolated chambers and a first surface, the second surface includes a plurality of capture agents, coupling of the substrate and the cover encloses each chamber of the plurality of chambers, and the dock is configured to releasably couple the substrate and the cover of the device. The system also includes, a first optics system comprising a first objective and configured to move along the X, Y, and Z axes (or at least one thereof), and produces at least one of a visible and a fluorescent light (preferably, in some embodiments, at least the fluorescent light, and can be a laser). The system also optionally includes a second optics system comprising a second objective and configured to moves along the X, Y, and Z axes (or at least one thereof), and produce an ablating laser beam, a first detector (e.g., a camera) optionally corresponding to the first optics system, and a processor configured to at least one of process information of the system and control the system or one or more components thereof. The first optics system may be positioned between the dock and the first detector.

Such embodiments may include one and/or another of the following additional steps, features, associated structure, functionality and/or clarification (and in some embodiments, a plurality thereof, and in still yet further embodiments, a majority or all thereof), yielding yet further embodiments of the current disclosure:
the laser (for either optics system) is selected from the group consisting of: a diode helium-neon, argon, xenon, nitrogen, carbon dioxide, carbon monoxide, hydrogen fluoride, deuterium fluoride, helium-cadmium, helium-mercury, helium-selenium, helium-silver, or neon-copper laser;
the detector comprises a digital camera, and/or the like;
the first optics system can be configured to expose the cover of the assembly to the fluorescent light so as to fluoresce formed complexes,
the detector can be configured to image the cover for the fluorescing complexes;
the detector can be configured to obtain a plurality of images of overlapping portions of both the first and second surfaces;
the second surface can be configured with a plurality of fiducial lines and the substrate is configured with a plurality of organized fiducial marks;
the processor can be configured, e.g., via computer instructions operating thereon configured to cause the processer to process image information so as to identify one or more chambers upon which fluorescence is captured in the images;
the processor can use the fluorescence and lines and marks (e.g., from the first and second surfaces) of the images to locate the one or more of the chambers;
the processor can be configured to assemble adjacent images together;
fiducial lines can be arranged on the second surface in a repeated, pre-defined pattern;
each fiducial line can be a unique color;
the fiducial marks can be are arranged on the first surface in-between columns, for example, of the chambers;
the marks can be configured with two or more different length of with equal spacing between;
and
chambers in between the spacing can be labeled.

These and other embodiments will become even more apparent with reference to the detailed description which follows, as well any associated figures corresponding thereto, a brief description of which is set out below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B are schematic, cross-sectional side views of a device for a cell selection system according to some embodiments of the present disclosure illustrated a first “unclamped/uncoupled” configuration (FIG. 1A) and a clamped/coupled configuration (FIG. 1B);

FIG. 2A is a top view of a ab-coated, cover/glass-slide portion of the device of FIGS. 1A-B, according to some embodiments of the present disclosure, which includes an enlargement of a portion of the slide;

FIG. 2B is a schematic perspective view of antibodies arranged on the slide of FIG. 2A relative to the substrate of the device of FIGS. 1A-B, according to some embodiments of the disclosure;

FIG. 2C is a schematic perspective view of the slide portion of FIGS. 1A-B, depicting, e.g., cytokine capture and identification of captured cell components, according to some embodiments, the perspective view also illustrating the well/chamber pattern;

FIGS. 3A-D are schematic side views of a device/assembly/chip according to some embodiments, depicting one process for identifying/selecting one or more cells in the chambers/well, according to some embodiments;

FIG. 4A is a schematic, cross-sectional side view depicting the configuration of the device of FIGS. 1A-B relative to a pair of light sources, according to some embodiments of the present disclosure.

FIG. 4B is a schematic, cross-sectional view of a single chamber of the substrate of the device of FIGS. 1A-1B, according to some embodiments, illustrating the uncoupling of the glass slide therefrom, and illustrating a single cell in the chamber;

FIG. 4C is a schematic, cross-sectional view of a single chamber of the substrate of the device of FIGS. 1A-1B, according to some embodiments, illustrating the open chamber, and nano-pipetting of the single cell out of the chamber;

FIG. 4D is a schematic, cross-sectional view of a single chamber of the substrate of the device of FIGS. 1A-1B, according to some embodiments, illustrating the open chamber, and using a mechanical force to push a cell out of a chamber;

FIG. 5 is a schematic, cross-sectional view of a single chamber of the substrate of the device of FIGS. 1A-1B, according to some embodiments, illustrating the open chamber, and nano-pipetting of the single cell out of the chamber, which, in some embodiments, includes a real-time imaging system; and

FIG. 6A is a schematic, cross-sectional view of a multiple chamber device, including chambers (which may also be referred to as wells, the terms well and chamber being used interchangeable throughout) which can accommodate a population of cells, according to some embodiments of the present disclosure.

FIG. 6B is a schematic, cross-sectional view of a multiple chamber device, including chambers (which may also be referred to as wells, the terms well and chamber being used interchangeable throughout) which accommodate individual cells, as well as multiple ports to inject such cells (or a fluid containing such cells), according to some embodiments of the present disclosure.

FIGS. 7A-C are schematics of various indicia/fiducial lines provided on a surface of a cover/glass-slide and substrate surface (FIG. 7C), for the device of FIGS. 1A-B (for example), according to some embodiments. FIGS. 7A-B illustrates example fiducial lines of the cover/glass-slide, and FIG. 7C illustrating image capture of fiducial lines and fiducial marks, according to some embodiments.

### DETAILED DESCRIPTION OF AT LEAST SOME OF THE EMBODIMENTS

The disclosure provides systems and methods configured to, in some embodiments, at least one of, and in some embodiments, a plurality of, and in some embodiments, all of the following.
flowing or otherwise arranging a single cell in a single well/chamber (and in some embodiments, a population of cells within a single well), among a plurality of wells/chamber on substrate of a device according to some embodiments;
providing materials within the well so as to enable the cells to continue at least normal cellular activity so as to, for example, express a protein and the like;
coupling the substrate with a cover (e.g., a glass slide), forming a device/assembly, where the slide includes antibodies/capture agents arranged (according to some embodiments) in a prearranged pattern so that at least some antibodies are arranged over each well/chamber (the foregoing may also be referred to as an antibody panel);
incubating the cells/wells so that the cell (or cells) produce cellular components (e.g., proteins, cytokines, and/or the like);
the cellular components can be configured to bind with the associated antibodies arranged over a respective well, forming complexes;
the cellular components bind with the capture agents adjacent each well/chamber;
an optics system and/or the like can direct a light from a light source into the glass slide so as to generate fluorescence of formed complexes;
the glass slide is imaged to capture indicia/fiducial lines/markings on one or both of a surface of the glass slide, and a surface of the substrate to identify at least one of an preferably all of fluorescing complexes and the indicia/fiducial lines/markings,
processing one or more images to identify specific wells/chambers which correspond to fluorescing complexes; and
at least one of collecting cells or cellular components of the identified wells/chambers or unidentified (i.e., wells/chambers which do not include fluorescing complexes), and ablating/eliminating cells or cellular components of the identified or unidentified wells/chambers.

The binding of the cellular components of the cells to the associated antibodies/agents enables at least one of measuring, identifying, eliminating, collection and/or removing one ore more particular cells (or population of cells) from the wells/chambers. Such embodiments enable a highly multiplexed reaction involving, for example, thousands of single cell reactions with hundreds of unique capture agents. In some embodiments, the resulting complex of the binding of at least one cellular components to an associated antibody fluoresces upon exposure to a particular wavelength(s) of light [US patent teaching fluorescence].

For example, in some embodiments, the devices, systems and methods of the present disclosure can simultaneously measure a plurality (e.g., 42) key effector proteins at the single cell level. Thus, some embodiments of the present disclosure may be used, for example, directly in pipeline drug development and/or CAR-T assessment by large-scale developers of cell-based therapeutics. The multiplexed parameters measured by the devices, systems and methods of the present disclosure can cover, for example, a complete range of relevant immune effector functions including stimulatory, pro-inflammatory, regulatory (negative), chemo-attractive, pro-growth and cytolytic (effective). Moreover, once a particular cell type or behavior is identified, some embodiments of the present disclosure can be configured to sort/select desired cells intended for subsequent collection from undesired cells intended for ablation/elimination. Because, in some embodiments, each cell is isolated in an enclosed chamber, cellular ablation may be accomplished by, for example, contacting a laser to any surface or any volume of fluid/component in the chamber including the cell itself (although, in some embodiments, contact with the cell is not required to lyse the cell when it has been identified for removal). Following the destruction of undesired cells, the remaining desired cells may be collected as a population or as individual cells from their respective chambers via nano-pipette and either stored individually or pooled to form a homogeneous population of cells. Alternatively, the releasably coupled substrate/cover are slightly separated, and a collection fluid is flowed across the substrate in sufficient volume to dislodge intact cells within the chambers and the fluid containing the homogeneous population of cells is collected for further analysis or processing.

Lasers used for cellular ablation, according to some embodiments, can include but are not limited to diode helium-neon, argon, xenon, nitrogen, carbon dioxide, carbon monoxide, hydrogen fluoride, deuterium fluoride, helium-cadmium, helium-mercury, helium-selenium, helium-silver, or neon-copper lasers. In some embodiments, laser-based cellular lysis is performed by directing the laser to contact (which can also be used to illuminate) the chamber which holds the cell identified for lysis. In some embodiments, cellular lysis via the laser of the disclosure does not require focusing the laser on the individual cell to be lysed. The lysis laser used to ablate a cell in a given chamber need not contact or illuminate adjacent chambers rendering the cells in adjacent chambers intact, healthy and otherwise unperturbed.

Accordingly, cells identified to have expression levels of one or more proteins, peptides, polypeptides, nucleic acids, small-molecules, and macromolecules above a pre-determined threshold can be collected. The releasably coupled device (e.g., the substrate comprising chambers and cells and the cover/glass-slide/surface comprising a plurality of capture agents), can be separated. Individual cells can then be collected from their respective chambers via nanopipette and either stored individually or pooled to form a homogeneous population of cells.

In certain embodiments, cells identified to have expression levels of proteins, peptides, polypeptides, nucleic acids, small-molecules, and macromolecules above the pre-determined threshold were collected. The releasably coupled substrate comprising chambers and cells and the surface comprising a plurality of capture agents were separated. A collection fluid was flowed across the substrate in sufficient volume to dislodge intact cells within the chambers and the fluid containing the homogeneous population of cells was collected for further analysis or processing.

Accordingly, in some embodiments, a device (and with other elements, a system) for the multiplexed detection of at least one, and in some embodiments, a plurality of compounds from single cells is provided and includes an array (which may also be referred to as a substrate), which includes a plurality of chambers releasably coupled to a cover or panel of capture agents (which may be a glass slide). Preferred capture agents include antibodies, however, capture agents may include any detectable entity that specifically binds to a cellular component of the disclosure. The detectable entity may comprise a detectable label, for example. Detectable labels may include, but are not limited to fluorescent labels.

The chambers (which may also be referred to as wells, such terms being used interchangeably throughout, although wells may generally refer to the recesses in the substrate, and chambers, the recesses enclosed by the cover) are preferably distributed on the substrate in a uniform arrangement, and, in some embodiments, each includes a length of greater than 50 μm and, optionally, may be configured to contain an isolated single cell in a nano-liter volume of contents (in some embodiments, a sub-nano-liter volume). The number of chambers can be any number, including, for example: 1, 2, 6, 12, 24, 48, 96, 384 or 1536 chambers, including arrays of 10 chambers, 100 chambers, 1000 chambers, 10,000 chambers, 100,000 chambers, 1,000,000 chambers or any number of chambers in between.

Each chamber, in some embodiments, includes a plurality of surfaces, between 2 and 8 (depending upon the embodiment), and may be curved, or faceted (e.g., multi-surfaced, with at least one surface being a “bottom” and at least one surface being a “side”. As noted above, the substrate with the array of wells can be releasably coupled to a cover, which is an additional surface (e.g., a “top” to the chamber), thereby enclosing the chamber on all sides. In some embodiments, the chamber can be configured as a rectangle and comprises or consists of at least five surfaces where the at least one surfaces is a bottom and at least one surface is a side.

Capture agent panels, according to some embodiments, comprise a plurality of immobilized capture agents, each immobilized capture agent capable of specifically binding to one of the plurality of cellular components. Preferably, the immobilized capture agents are arranged in uniform capture agent panels. Preferably, the immobilized capture agents are attached to a surface in a repeatable pattern, wherein each repeat of the pattern aligns with a chamber of the plurality of chambers. The array and capture agent panels are coupled to form a plurality of enclosed volumes (see above), each enclosed volume comprising a chamber and a capture agent panel such that the contents of each chamber are accessible to each and every capture agent of the capture agent panel.

Chambers of the array according to some embodiments, may take on any shape and may have any dimension, however, in some embodiments, the array comprises at least 2 chambers, but may also include 5, 10, 15, 20, 25, 50, 100, 150, 500, 1000, 1500, 2000 or any integer between of chambers. Each chamber may have a depth/height of between 1 μm and 2000 μm, a diameter of between 1 μm and 2000 μm, a width of between 1 μm and 2000 μm and/or a length of between 1 μm and 2000 μm. The distance between any two chambers of the array may be between 1 μm and 2000 μm. In some embodiments, at least one chamber comprises a high aspect ratio rectangular well, having dimensions of about 1-2 mm in length and about 5-50 μm in depth. In some embodiments, each chamber is rectangular with a length of about 10-2000 μm, a width of about 10-100 μm, and a depth of about 10-100 μm.

In some embodiments, the capture agent panel may comprise between 3 and 50 different capture agents, thereby allowing for the detection of between 3 and 50 different cellular components (for example), but may include greater than 10 different capture agents, thereby allowing for the detection of greater than 10 different cellular components, or may comprise greater than 42 different capture agents, thereby allowing for the detection of greater than 42 different cellular components, or may comprise greater than 50, 100, 150, 200, 250, 300, 350, 400, 450, 500 or any number in between of different capture agents, thereby allowing for the detection of greater than 50, 100, 150, 200, 250, 300, 350, 400, 450, 500 or any number in between of different cellular components. In certain embodiments, the array comprises a chamber density of about 200 micro-chambers per cm2 to about 20,000 micro-chambers per cm2.

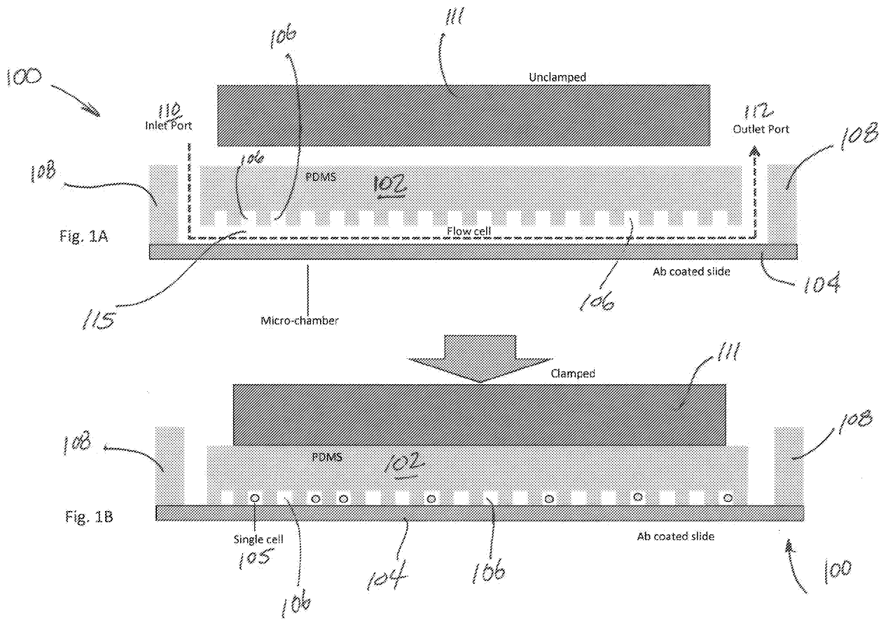

Accordingly, as shown in FIG. 1A-1B, a device 100 includes substrate 102 and a cover 104, which in some embodiments comprises a glass slide. The substrate can be comprises of PDMS (Polydimethylsiloxanes), and can be configured to include a plurality of chambers 106 (which can be arranged in an organized pattern). Also included are side-walls 108, which surround the substrate 102, which, when coupled or otherwise clamped via dock/clamp 111 to the cover 104, for an inlet 110 (which may also be referred to as an inlet port), and an outlet 112 (which otherwise may also be referred to as an outlet port). A flow of cells can be established to flow from the inlet to the outlet prior to the coupling of the substrate 102 to the cover 104, thus establishing a channel 115. The spacing of the uncoupled substrate 102 and cover 104 is such that only a single line of cells promulgate therebetween. Accordingly, the chamber(s) 106, according to some embodiments, are sized so that they can only accommodate a single cell 105, as well as, in some embodiments, fluids and/or other materials to retain the cell in a viable condition so as to produce cellular components. Thus, when the substrate and cover are coupled/clamped together, the configuration causes each cell to be captured by a respective well (along with, in some embodiments, the materials necessary to sustain the cells within the well, which can be present in the flow as well). The clamped/coupled configuration is shown in FIG. 1B.

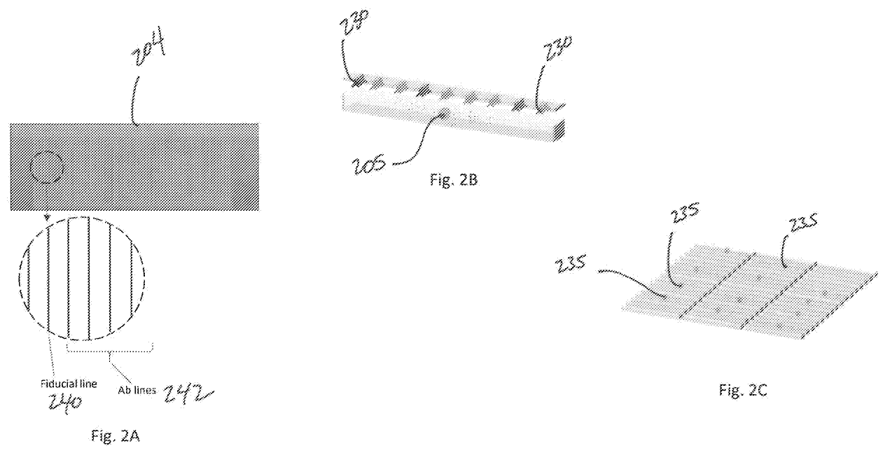

FIGS. 2A-C illustrate various schematic views of the cover 104 from FIGS. 1A-B, which is referred to in FIGS. 2A-C as cover 204. In FIG. 2A, the cover 204 is configured with a spatial arrangement of capture agents/antibodies 230, to establish a high-plex agent/antibody (Ab) panel. Such an arrangement as noted above, is in a particular pattern (in some embodiments), including fiducial lines 240 corresponding to capture agent positions/lines 242 so that the antibodies can match up to individual wells in the substrate. The particular pattern can be stored in a database/look-up table such that the well/cell which produced cellular components that bounded to a particular antibody (the location of which would be known), could be easily identified.

Accordingly, FIG. 2B illustrates a perspective view of a row of wells and the portion of the cover with antibodies 230. Finally, FIG. 2C is a perspective view of the cover 204 (e.g., glass slide), illustrating the well/chamber pattern, to which certain cells which produced cellular components to which identified antibodies of the cover bind with, are bound to the capture agents/antibodies (shown as ref. numeral 230). Specifically, and for example, the cover/slide 204 is analyzed for, e.g., protein, cytokine capture by the capture agents/antibodies 230, and cells 205 of interest are identified (identified cells/wells/chamber 235).

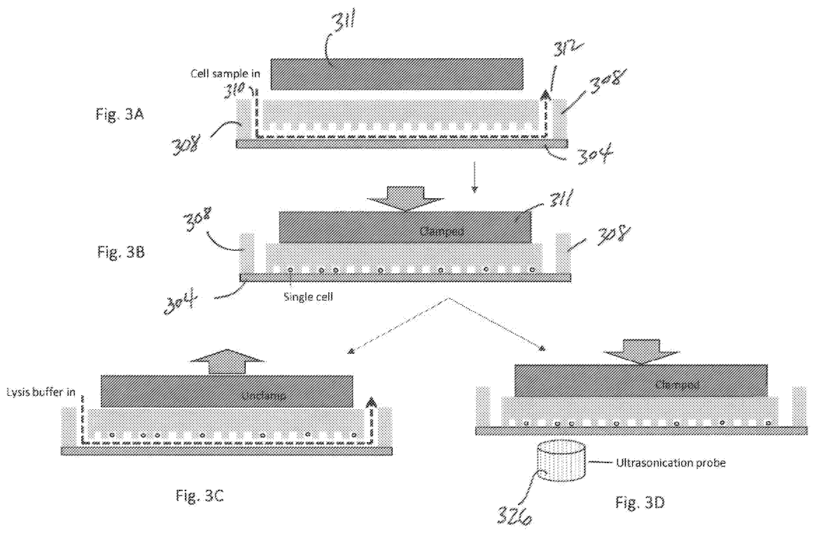

FIGS. 3A-3D correspond to a process, according to some embodiments of the present disclosure, for identifying cells, using the device/system according to FIGS. 1A-B. Accordingly, in FIG. 3A, a cell sample (which, in some embodiments, includes a volume of fluid necessary for the cells to express one or more proteins, for example) is loaded into device 300, via inlet port 310. The cell sample is flowed from the inlet to the outlet 312. Thereafter, the substrate 302 and cover 304 are coupled together to form an assembly, via a docking device 301, as shown in FIG. 3B, which may comprise a clamp 311. At least some of the reference numbers in FIGS. 3A-3D correspond to those of FIGS. 1A-1B, except to lead with a “3”.

After a period of time, the assembly is unclamped, as shown in FIG. 3C, and slowly flow in lysis buffer is flowed into the device from the inlet 310 to the outlet 312, the buffer configured to release intracellular contents from cell (i.e., a lysis buffer). The cells are held in place by at least one of gravity (assembly “upside down”), centrifuge force, and a hydrophilic surface marker specific coating on the cover surface. Alternatively, as shown in FIG. 3D, while the device is coupled/clamped, and a physical method to lyze the cells is applied (e.g., an ultrasonication field by an ultrasound device 326, and/or UV exposure via a UV source). The means for producing the physical force, in some embodiments, can be placed adjacent the cover/slide. In both procedures, thereafter, the cover/slide captures released intracellular components (e.g, phosphoproteins, metabolites), where thereafter may be analyzed (in some embodiments).

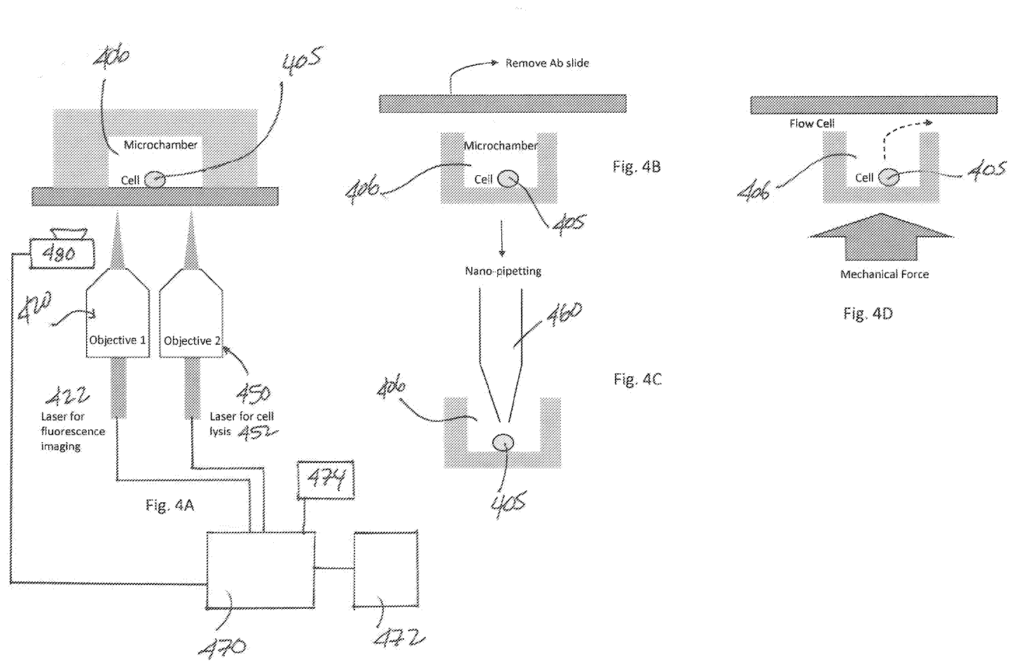

FIG. 4A is a schematic, cross-sectional side view depicting the configuration of the device of FIGS. 1A-B relative to a pair of light sources, according to some embodiments of the present disclosure. As shown, a first optics device/system 420 (which may be referred to Objective I), which includes a fluorescence light source 422 (e.g., a first laser), is configured to direct light through the cover (e.g., glass slide), so as to fluoresce the complex (i.e., the cellular component from the well bound the antibody on the cover) in a respective well in the device 400. The first optics system 420 can also include an imaging system 480 to image the fluorescent complex, or detect the complex or image or detect a secreted or intracellular component. A second optics device/system 450 (which may be referred to Objective II), which includes a laser 452 for lysis of the cell in a particular well(s) through the cover. Both optics systems are configured to move in three-dimensions (according to some embodiments), and one or two dimensions (according to some embodiments). In some embodiments, any of the imaging functionality/systems/device can be or include a digital camera and/or a digital scanner, so as to obtain, for example, scanned fluorescent images of the surface (e.g., comprising a plurality of capture agents/complexes). For example, two color channels, 488 (blue, PMT 350, Power 90) and 635 (red, PMT 600, Power 90) can be used to collect fluorescence signals. Obtained images can then be exported and processed to quantify expression levels of components of the cellular secretome (i.e., cellular components).

FIG. 4B is a schematic, cross-sectional view of a single chamber 406 of the substrate 402 of the device of FIGS. 1A-1B, according to some embodiments, illustrating the uncoupling of the cover 404 therefrom, and illustrating a single cell 405 in the chamber 406. As shown in FIG. 4C, the single cell can be pipetted out of the cell (via, for example, a nano-pipet 460 of 1-100 nanoliters, for example). In some embodiments, instead of removing the cover completely from the device, it may be simply spaced away from the substrate (at a distance configured to enable movement of cells into the channel, and mechanical forces applied to the substrate (e.g., in some embodiments, at least one of a micro-puncher, a pulsed laser). In such embodiments, as shown in FIG. 4D, the mechanical forces can be used to selectively push a cell out of from a respective well, to flow out through the device outlet (for example).

A processor or controller (terms used interchangeably) 470 can be included to at least one of control one or more components of the systems (e.g., optics systems, lasers, light sources, imagers, interfaces, output and input devices), and process information. The processor may be therefore in communication (wired or wireless) to various components, including, for example, an user interface 472 and an output means 474. The processor can also be configured to communicate with a computer network (e.g., an intranet or internet) for access. At least some of the reference numbers in FIGS. 4A-4D correspond to those of FIGS. 1A-1B, except to lead with a “4”.

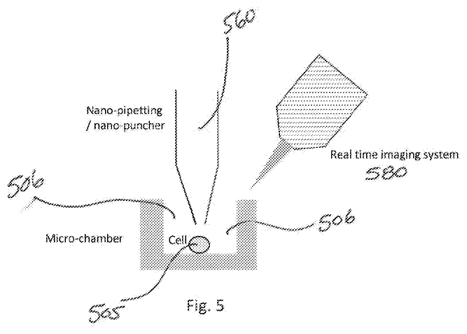

FIG. 5 illustrates a schematic, cross-sectional view of a single chamber 510 of the substrate 502 of the device 500 of FIGS. 1A-1B, according to some embodiments, illustrating the open chamber 510, and nano-pipetting 511 of the single cell 505 out of the chamber (similar to FIG. 4C), as well as a real-time imaging system 580. Such a real-time imaging system can be mounted relative to the device of FIGS. 1A-B, so as to be movable in three (or, in some embodiments, less than three) dimensions. Preferably, the real-time imaging system can be configured with, for example, 1 micron resolution. At least some of the reference numbers in FIG. 5 correspond to those of FIGS. 1A-1B, except to lead with a “5”.

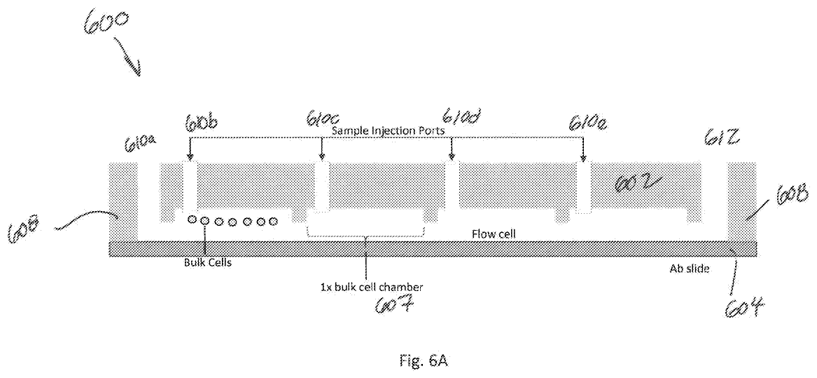

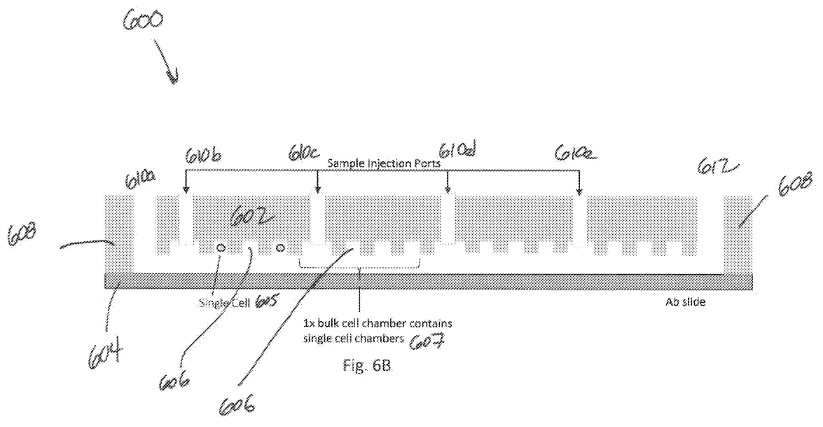

FIG. 6A is a schematic, cross-sectional view of an uncoupled, multiple chamber device 600, including wells which can accommodate a population of cells, according to some embodiments of the present disclosure, which is similar to FIGS. 1A-1B. Accordingly, substrate 602 (e.g., PDMS) and a cover 604, which in some embodiments, comprises a glass slide. The substrate includes a plurality of bulk chambers 607 (which can be arranged in an organized pattern), and side-walls 608, which surround the substrate 602, which, when coupled or otherwise clamped to the cover 604, for an inlet 610a (which may also be referred to as an inlet port), and an outlet 512 (which otherwise may also be referred to as an outlet port). A flow of cells can be established to flow from the inlet to the outlet prior to the coupling of the substrate 502 to the cover 504. The embodiments illustrated in FIG. 6A includes multiple sample injection ports 610b-e, one of which are provided at one the ends (the other being an outlet/port 612), and four (one or a plurality) along the distance of the substrate (although one or more of any of the ports can be inlets or outlets). FIG. 6B corresponds to the embodiments of FIG. 6A, including multiple sample injection ports 610b-e, but also includes bulk wells/chambers 607 which also include single cell chambers 606 to accommodate only a single cell.

The spacing of the uncoupled substrate 602 and cover 604 (which is also applicable to the embodiments shown in FIGS. 1A-5 as well) is such that only a single line of cells promulgate therebetween. Accordingly, the wells, according to some embodiments, are sized so that they accommodate a determined number of cells, as well as, in some embodiments, fluids and/or other materials to retain the cell in a viable condition so as to produce cellular components. Thus, when the substrate and cover are coupled/clamped together, the configuration causes the predetermined number of cells to be captured by a respective well (along with, in some embodiments, the materials necessary to sustain the cells within the well, which can be present in the flow as well).

In some embodiments, a highly multiplexed method is provided (and associated device/system, examples of which are noted above and throughout the disclosure) for evaluating, for example, the secretome of individual cells in a functionally heterogeneous population following contact with a target cell or a stimulatory agent. To this end, analysis of the composition of the secretome of a subject cell may be used to determine at least one of the subject cell's: identity, viability, safety and efficacy, when used in a cell-based therapy. Cellular therapies may include autologous or allogeneic cells. Cellular therapies may include modified cells, including, but not limited to T-cells that express at least one artificial or chimeric antigen receptor.

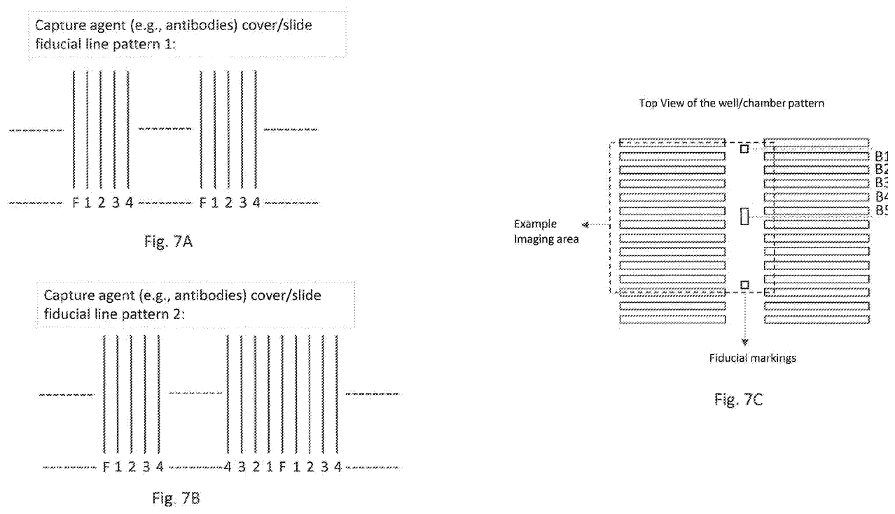

Some embodiments of the highly multiplexed process are explained with reference to FIGS. 7A-C. Fiducial lines are printed (or otherwise provided) in a repeated, pre-defined pattern (e.g., fiducial lines F-1-2-3-4); see e.g., FIG. 7A. Each fiducial line can include a unique color (e.g., green, red, labeled by, for example, a coloring dye and/or a florescence dye). Once a fiducial line can be identified by its unique color, the remainder of the lines, both before and after (in some embodiments, they can be the same color as the fiducial, while in other embodiments, they can be a different color), the associated content of a chamber can be identified (upon binding of cellular components thereto); see, e.g., FIG. 7B. Accordingly, for a single-cell-barcode chip (SCBC) pattern, the line order before and after a fiducial line are inversed.

In some such embodiments, one or more fiducial marks, dots, or dashes (such terms used interchangeably with respect to fiducial marking) are arranged or otherwise provided/printed in-between columns of the chambers of the substrate (FIG. 7C). In some embodiments, two or more different length of dots with equal spacing between (in some embodiments), e.g., dot, dash, dot, dash (e.g., similar to Morse Code). Chambers in between this spacing can be then labeled (e.g., A1, B1, similar to a spreadsheet). When imaging the assembly (in particular, from the glass slide to view both the fiducial lines and fiducial lines, according to some embodiments), the method and system is configured so that each image includes at least two (2) of the fiducial dashes, images can be stitched together independently from the chambers, which are identical to each other. Therefore, specific chambers containing specific cells can easily be identified and/or tracked, relative to a combination of the fiducial lines and dots/dashes.

One of skill in the art will appreciate that the immediately previous patterning methodology is not limited to antibody coatings, indeed, the methodology can also be used with, for example, DNA probe coatings for DNA/RNA capture and proximity ligation assays.

Accordingly, imaging of the assembly via the slide using imaging means (e.g., digital camera, in some embodiments, for example, see, e.g., FIG. 4A, and first/concurrently exposing the Ab slide to a fluorescing light source (e.g., a laser of known wavelengths associated with fluorescing the complexes formed between the antibodies and the cells/cellular components thereof), and then, via an imaging means (digital camera and the like), imaging the Ab slide, wells/chamber having cells upon which cellular components bound to the antibodies can be determined. This can be accomplished by using the fiducial lines and markers, discussed above, the locations and patterning of the antibodies thereof, all provided to a processor. The locations and patterning of the known antibodies, and their location relative to the wells/chambers of the substrate are stored in a lookup table, which itself is stored in a memory accessible to the processor (or part of the processor). The processor includes instructions operating effective to cause the processor to process the images, access the information in the lookup tables, to identify the wells/chambers that had cellular components which formed complexes with specific known antibodies of the Ab slide, so as to at least one of retrieve/collect specific cells, and/or eliminate other cells (e.g., ablating them).

In some embodiments of the disclosure, a stimulus is applied to a well so that a subject cell produces one or more cellular components. In some embodiments, the stimulus is another cell which actually contacts the cell within the well. In the event that each chamber comprises a subject cell and a target cell, the target cell may be operably linked to a surface or to a component of the chamber, such that, the collection step comprises capturing the subject cell but not the target cell because, in some embodiments, the target cell cannot exit the chamber by flowing into a flow channel. In some embodiments, the subject cell and the target cell are distinctly and detectably labeled (or a cell-surface marker on the subject cell and a cell surface marker on the target cell are distinctly and detectably labeled), and upon viewing the subject cell and target cell under either visible or fluorescent light, the subject cell may be selectively captured as an individual cell, by, for example, a pipette or a nano-pipette.

For example, in some embodiments, a method of identifying a secretome from a subject cell within a heterogeneous cell population is provided and comprises contacting the subject cell and a target cell or a stimulatory agent in at least one chamber of a plurality of chambers arranged within a substrate, where the chamber is in fluid communication with an antibody panel which is removably attached to the chamber. The method also includes maintaining the subject cell and the target cell and/or the stimulatory agent in the chamber under conditions sufficient to permit the subject cell to secrete at least one of a peptide, polypeptide, and protein, and at least one antibody of the antibody panel specific for the at least one protein to bind the at least one peptide, polypeptide, or protein, forming at least one of an antibody:secreted peptide, antibody:secreted polypeptide, or an antibody:secreted protein complex. The method also includes decoupling or otherwise removing the antibody panel from the substrate, and imaging the at least one of the peptide, polypeptide, or protein, forming at least one of an antibody:secreted peptide, antibody:secreted polypeptide, or an antibody:secreted protein complex. By imaging, secretome of the subject cell can be identified when the subject cell contacts the target cell or the stimulatory agent.

In some embodiments, a method of identifying a secretome from a subject cell within a heterogeneous cell population is provided and comprises contacting the subject cell and a target cell or a stimulatory agent under conditions sufficient to permit stimulation of the subject cell, introducing the subject cell to at least one chamber of a plurality of chambers arranged in a substrate, where each chamber is in fluid communication with an antibody panel (see above) and the antibody panel is removably attached/coupled to the chamber/substrate. The method further includes maintaining the subject cell in the chamber under conditions sufficient to permit the subject cell to secrete at least one of a peptide, polypeptide, and protein, and at least one antibody of the antibody panel specific for the at least one protein to bind the at least one peptide, polypeptide, or protein, forming at least one of an antibody:secreted peptide, antibody:secreted polypeptide, or an antibody:secreted protein complex. The method additionally includes removing the antibody panel from the chamber, and imaging the at least one peptide, polypeptide, or protein, forming at least one of an antibody:secreted peptide, antibody:secreted polypeptide, or an antibody:secreted protein complex. Imaging allows identification of the secretome of the subject cell following contact with the target cell or the stimulatory agent. In some embodiments, the method further includes at least one of: disrupting contact between the subject cell and the target cell or the stimulatory agent, and the subject cell and the target cell or the stimulatory agent are comprised by a composition. In some embodiments, the subject cell and the target cell or the stimulatory agent are in fluid communication, the subject cell and the target cell or the stimulatory agent are comprised by a composition. In such embodiments, the subject cell and the target cell or the stimulatory agent are in fluid communication, and the method further comprises the step of depleting the target cell or the stimulatory agent from the composition.

In some embodiments, the heterogeneous cell population is a functionally heterogeneous cell population, where the functionally heterogeneous cell population may comprise at least two cells that produce a secretome in response to a stimulus, and the first cell produces a first secretome, and the second cell produces a second secretome. In some embodiments, the first secretome and the second secretome are not identical. In some embodiments, the secretome comprises one or more distinct peptide(s), polypeptide(s), or protein(s) that indicate diminished or decreasing cell function or cell viability.

Secretomes of the disclosure may comprise one or more peptides, polypeptides, proteins, small molecules, and/or ions. When the secretome comprises a small molecule or an ion, detectable labels may be used in addition or in place of antibodies to identify, quantify, or otherwise analyze the small molecule and ions of the secretome. In addition, secretomes of the disclosure may be released from a subject cell actively or passively. For example, secretomes of the disclosure may be released from a subject cell via a vesicle, an intercellular gap junction and/or a transmembrane channel or pump. In some embodiments, the secretome comprises one or more distinct peptides, polypeptides, or proteins that indicate augmented or increasing inflammation, and/or increased cell activity or cellular stimulation.

In some embodiments, the functionally heterogeneous cell population comprises one or more immune cells, where the one or more immune cells can comprise a T-lymphocyte, a B-lymphocyte, a natural killer (NK) cell, a macrophage, a neutrophil, a mast cell, an eosinophil, or a basophil. In certain embodiments, the T-lymphocyte comprises a nai:ve T-lymphocyte, an activated T-lymphocyte, an effector T-lymphocyte, a helper T-lymphocyte, a cytotoxic T-lymphocyte, a gamma-delta T-lymphocyte, a regulatory T-lymphocyte, a memory T-lymphocyte, or a memory stem T-lymphocyte. In some embodiments, the T-lymphocyte expresses a non-naturally occurring antigen receptor. In certain embodiments, the T-lymphocyte expresses a Chimeric Antigen Receptor (CAR).

In some embodiments, the functionally heterogeneous cell population comprises one or more immune cells, where the one or more immune cells can comprise a T-lymphocyte, a B-lymphocyte, a natural killer (NK) cell, a macrophage, a neutrophil, a mast cell, an eosinophil, or a basophil. In some embodiments, the B-lymphocyte comprises a plasmablast, a plasma cell, a memory B-lymphocyte, a regulatory B cell, a follicular B cell, or a marginal zone B cell.

In some embodiments, the subject cell is an immune cell, which can comprise a T-lymphocyte, a B-lymphocyte, a natural killer (NK) cell, a macrophage, a neutrophil, a mast cell, an eosinophil, or a basophil. In certain embodiments, the T-lymphocyte comprises a nai:ve T-lymphocyte, an activated T-lymphocyte, an effector T-lymphocyte, a helper T-lymphocyte, a cytotoxic T-lymphocyte, a gamma-delta T-lymphocyte, a regulatory T-lymphocyte, a memory T-lymphocyte, or a memory stem T-lymphocyte. In some embodiments, the T-lymphocyte expresses a non-naturally occurring antigen receptor, and the T-lymphocyte can express a Chimeric Antigen Receptor (CAR).

In some embodiments:
the subject cell is an immune cell, which can comprise a T-lymphocyte, a B-lymphocyte, a natural killer (NK) cell, a macrophage, a neutrophil, a mast cell, an eosinophil, or a basophil;
the B-lymphocyte can comprise a plasmablast, a plasma cell, a memory B-lymphocyte, a regulatory B cell, a follicular B cell, or a marginal zone B cell;
the functionally heterogeneous cell population (see above), or subject cell, can comprise:
   one or more neuronal cells (e.g., a neuron, a glial cell, an astrocyte, a satellite cell, or an enteric glial cell);
   one or more endocrine cells (e.g., isolated or derived from a pineal gland, a pituitary gland, a pancreas, an ovary, a testicle, a thyroid gland, a parathyroid gland, a hypothalamus, or an adrenal gland);
   one or more exocrine cells (e.g., isolated or derived from a salivary gland, a sweat gland or a component of the gastrointestinal tract—which may comprise a mouth, a stomach, a small intestine, and a large intestine).

In some embodiments, the step of contacting the subject cell and the target cell (or the stimulatory agent) in a chamber comprises:
direct contact of the subject cell and the target cell or the stimulatory agent;
indirect contact of the subject cell and the target cell or the stimulatory agent (e.g., fluid communication between the subject cell and the target cell or the stimulatory agent, communication between the subject cell and the target cell or the stimulatory agent through a natural or artificial extracellular matrix, where communication between the subject cell and the target cell or the stimulatory agent can be through an intermediate cell).

In some embodiments, the target cell is at least one of:
a cancer cell (e.g., a primary cancer cell, which can be a metastic cancel cell) or a cultured cancer cell, a B-lymphocyte,
a bacteria, yeast, or microbe, an infected cell (e.g., infected cells that may have contacted or may have been exposed to a virus, bacteria, yeast, or microbe), and
a host cell (e.g., any cell isolated or derived from the same individual as the subject cell; in some embodiments, the host cell perpetuates an autoimmune response).

The term “functional” cell is meant to describe a viable cell that does not contribute to a disease or disorder in the host. Alternatively, or in addition, the term “functional” cell may describe a cell without any known mutations that cause a disease or disorder in the host. For example, a functional cell may be non-cancerous and/or non-autoimmune.

In some embodiments, the stimulatory agent comprises a stimulatory antibody which can be a monoclonal antibody, which can be:
a fully human antibody,
a humanized antibody,
a chimeric antibody,
a recombinant antibody or a modified antibody, e.g.: one or more sequence variations when compared to a fully human version of an antibody having the same epitope specificity, one or more modified or synthetic amino acids, or a chemical moiety to enhance a stimulatory function; the stimulatory antibody specifically binds—in some embodiments—an epitope of a T cell regulator protein (in some embodiments, the T cell regulator protein comprises programmed cell death protein I (PD-I)), and a Nivolumab or a biosimilar thereof.

In some embodiments, the stimulatory agent comprises a stimulatory ligand (e.g., a programmed death ligand I (PD-LI)).

In some embodiments:
each antibody of the antibody panel is attached to a surface that is removably attached to the chamber;
each antibody of the antibody panel is attached to the surface to form a repeating pattern, and in some embodiments, each chamber of the plurality of chambers of the substrate comprises a repeat of the pattern;

Antibody panels, according to some embodiments, form patterns in which each repeat comprises a full panel of designated antibodies. For example, if the antibody panel comprises antibodies “a”, “b” and “c”, then each repeat of the pattern also comprises at least one of antibody “a”, “b” and “c”. The pattern need only have a size scale such that each chamber aligns with at least one repeat of the pattern. In some embodiments, the pattern need only have a size scale such that each chamber aligns with one repeat of the pattern. When additional detectable labels are added to the panel to identify, capture or quantify secreted small molecules and/or ions, the detectable labels also repeat by the same rules set out for the antibody pattern.

In some embodiments, the conditions sufficient to permit at least one of the subject cell to secrete at least one of a peptide, polypeptide, and protein, and at least one antibody of the antibody panel specific for the at least one protein to bind the at least one peptide, polypeptide, or protein, forming at least one of an antibody:secreted peptide, antibody:secreted polypeptide, or an antibody:secreted protein complex, may comprise 5% CO2 and 37° C. for a period of 2 hours, about 2 hours or at least 2 hours. Alternatively, the period may be 4 hours, about 4 hours or at least 4 hours; 8 hours, about 8 hours or at least 8 hours; 12 hours, about 12 hours or at least 12 hours; 16 hours, about 16 hours or at least 16 hours; or 24 hours, about 24 hours or at least 24 hours. In certain embodiments, the period is 16 hours, about or at least 16 hours.

In some embodiments, at least one chamber of the plurality of chambers of the substrate comprise cell media that maintains the viability of the subject cell for multiple steps (e.g., from contacting the subject cell and target cell or the stimulatory agent through removal of the antibody panel comprising antibody complexes with one or more of a peptide, polypeptide or protein secreted from the subject cell).

In some embodiments, a step of determining a Polyfunctional Strength Index (PSI) is provided (which can be included as a step in one and/or another of the disclosed method embodiments). Polyfunctionality is a measure of efficacy and potency of cells intended for cellular therapy. Of particular value is the polyfunctional strength index (PSI), which is a metric that factors in the polyfunctionality of cells in a sample, and the signal intensity of the cytokines secreted by each cell. It is found by multiplying the percentage of polyfunctional cells of a sample (single cells secreting two or more cytokines), by the average signal intensity of these cytokines. Additional information about PSI may be found at WO 2018/049418 (the contents of which are incorporated herein in their entirety).

Accordingly, the PSI, in some embodiments, is the product of a percentage of polyfunctional subject cells within the heterogeneous cell population, and an average signal intensity of two or more cytokines. The average signal intensity of two or more cytokines can be the average signal intensity of two or more distinct cytokines (i.e. AB versus AA). In some embodiments, the polyfunctional subject cells, at a single cell level, secrete at least two cytokines, which can be distinct cytokines (i.e. AB versus AA), or the same (e.g. AB and AB). In some embodiments, an increase in the PSI indicates an increase in the potency of the polyfunctional subject cells.

In some embodiments, a stimulated cell may exhibit a PSI that is 1×, 2×, 3×, 4×, 5×, 10×, 25×, 50×, 100×, or 1000× higher than that of a non-stimulated cell. The PSI can be further broken down into defined cytokine groups, illustrating the impact of particular groups of cytokines on the cell's polyfunctionality. In certain embodiments, effector cytokines are major drivers of the polyfunctionality, as they account for about 75% of the total PSI of a sample cell. It is possible to analyze the contribution of each individual cytokine to the overall PSI of a cell. Accordingly, in certain embodiments, 1, 2, 3, 4, 5, 6, 7, 8, 9, 10, or more cytokines can drive the polyfunctional strength of the sample relative to the overall secretome of a cell (although in some embodiments, other cytokines contribute to a lesser degree to the PSI of the sample).

In some embodiments, a secretome produced by one and/or another of the disclosed methods is used to identify a T-Lymphocyte expressing a CAR that specifically binds an antigen presented on a target cell, a CAR that specifically binds a stimulatory agent, or a CAR that specifically binds an antigen presented on a target cell and specifically binds a stimulatory agent;

In some embodiments, secretome produced by one and/or another methods of the present disclosures can be used to evaluate the safety of a cellular therapy, where the cellular therapy comprises the subject cell and:
the cellular therapy is intended to respond to the target cell or the stimulatory agent; the cellular therapy is considered safe when the secretome lacks one or more peptide(s), polypeptide(s), or protein(s) that at least one of: stimulates the immune system, indicates decreased cell viability, and indicates a selective response to the target cell;
the subject cell is a chimeric antigen receptor (CAR)-expressing T cell and the cellular therapy is intended to respond to the target cell or the stimulatory agent, where the target cell is a cancer cell that expresses an antigen to which the chimeric antigen receptor (CAR) specifically binds, and upon binding the antigen, the chimeric antigen receptor (CAR) stimulates the T cell. The cellular therapy is considered efficacious when the secretome comprises one or more peptide(s), polypeptide(s), or protein(s) that stimulate the immune system above a first threshold, where the one or more peptide(s), polypeptide(s), or proteins comprise one or more cytokines, and the one or more cytokines are selected from the group consisting of effector, stimulatory or chemoattractive cytokines. The cellular therapy is considered safe when the secretome comprises one or more peptides, polypeptides, or proteins that mediate a deleterious process, where the one or more peptides, polypeptide(s), or proteins comprise one or more cytokines, and where the one or more cytokines are selected from the group consisting of regulatory and inflammatory cytokines. In some embodiments of this use, the effector cytokines are selected from the group consisting of Granzyme B, IFN-y, MIP-1a, Performin, TNF-a, and TNF-.

In some embodiments of the above-noted use:
the stimulatory cytokines may be selected from the group consisting of GM-CSF, IL-12, IL-15, IL-2, IL-21, IL-5, IL-7, IL-8 and IL-9,
the chemoattractive cytokines may be selected from the group consisting of CCL-I 1, IP-10, MIP-1 and RANTES;
the regulatory cytokines may be selected from the group consisting of IL-10, IL-13, IL-22, IL-4, TGF-1, sCD137 and sCD40L;
the inflammatory cytokines may be selected from the group consisting of IL-1 7A, IL-1 7F, IL-I, IL-6, MCP-1 and MCP-4;
the deleterious process may comprise inflammation;
the deleterious process may comprise an autoimmune response; and/or
the deleterious process comprises a non-selective response to the target cell.

In some embodiments, a method of identifying a subject cell population as efficacious for use in an adoptive cell therapy, is provided and comprises detecting at least one component of a secretome of each subject cell of the subject cell population according to the method of identifying a secretome from a subject cell of the disclosure, identifying a subpopulation of polyfunctional cells of the subject cell population, where a polyfunctional cell of subject cells of the subject cell population secrete two or more signaling molecules, calculating a percentage of polyfunctionality of the subject cell population, where the percentage of polyfunctionality is the percentage of polyfunctional cells within the subject cell population, measuring a signal intensity of a first signaling molecule of the secretome of each polyfunctional cell of the subject cell population, measuring a signal intensity of a second signaling molecule of the secretome of each polyfunctional cell of the subject cell population, calculating a Polyfunctional Strength Index (PSI) for each polyfunctional cell of the subject cell population, where the PSI comprise (a) the product of the percentage of polyfunctionality of the subject cell population and the signal intensity of the first signaling molecule and (b) the product of the percentage of polyfunctionality of the subject cell population and the signal intensity of the second signaling molecule, identifying the subject cell population as efficacious for use in an adoptive cell therapy when the PSI indicates that at least 50% of the subject cells in the subject cell population are polyfunctional, the signal intensity of the first signaling molecule indicates that the concentration of the first signaling molecule within the chamber is between 2 pg/ml and 10,000 pg/ml, inclusive of the endpoints, and the signal intensity of the second signaling molecule indicates that the concentration of the second signaling molecule within the chamber is between 2 pg/ml and 10,000 pg/ml, inclusive of the endpoints. The method may also include identifying the subject cell population as not efficacious for use in an adoptive cell therapy when the PSI indicates that less than 50% of the subject cells in the subject cell population are polyfunctional, the signal intensity of the first signaling molecule indicates that the concentration of the first signaling molecule within the chamber is less than 2 pg/ml, and the signal intensity of the second signaling molecule indicates that the concentration of the second signaling molecule within the chamber is less than 2 pg/ml.

In some embodiments for identifying a subject cell population as efficacious for use in an adoptive cell therapy, the subject cell population comprises a plurality of immune cells which can be a T-lymphocyte, a B-lymphocyte, a natural killer (NK) cell, a macrophage, a neutrophil, a mast cell, an eosinophil, a basophil or a combination thereof. The T-lymphocyte may be a native T-lymphocyte, an activated T-lymphocyte, an effector T-lymphocyte, a helper T-lymphocyte, a cytotoxic T-lymphocyte, a gamma-delta T-lymphocyte, a regulatory T-lymphocyte, a memory T-lymphocyte, or a memory stem T-lymphocyte. Moreover, the T-lymphocyte may express a non-naturally occurring antigen receptor, and/or a Chimeric Antigen Receptor (CAR).

In some embodiments, identifying a subject cell population as efficacious for use in an adoptive cell therapy:
the subject cell population comprises:
   a plurality of neuronal cells (e.g., a neuron, a glial cell, an astrocyte, a satellite cell, an enteric glial cell or a combination thereof);
   a plurality of endocrine cells (e.g., one or more cells isolated or derived from a pineal gland, a pituitary gland, a pancreas, an ovary, a testicle, a thyroid gland, a parathyroid gland, a hypothalamus, or an adrenal gland); and/or
   a plurality of exocrine cells (e.g., one or more cells isolated or derived from a salivary gland, a sweat gland or a component of the gastrointestinal tract, where the component of the gastrointestinal tract comprises a mouth, a stomach, a small intestine, and a large intestine).
the target cell:
   is a cancer cell (e.g., a primary cancer/metastatic cell or a cultured cancer cell);
   is a B-lymphocyte;
   is a bacteria, yeast, or microbe;
   is an infected cell (e.g., cells which may have contacted or may have been exposed to a virus, bacteria, yeast, or microbe);
   is a host cell (e.g., any cell isolated or derived from the same individual as the subject cell; the host cell may or may not perpetuate an autoimmune response, and the host cell may be a functional cell);

In some embodiments, identifying a subject cell population as efficacious for use in an adoptive cell therapy:
the first signaling molecule is a peptide, a polypeptide, or a protein (e.g., a cytokine), and/or the second signaling molecule is a peptide, a polypeptide, or a protein (e.g., a cytokine);
the subject cell population comprises a plurality of T-lymphocytes, where the target cell is a cancer cell, an infected cell or a host cell that perpetuates an autoimmune response, the first signaling molecule comprises an effector cytokine, a stimulatory cytokine, or a chemoattractive cytokine, and the second signaling molecule comprises an effector cytokine, a stimulatory cytokine, or a chemoattractive cytokine. In some embodiments:
   at least one T-lymphocyte of the plurality of T-lymphocytes may express a chimeric antigen receptor (CAR), where each T-lymphocyte of the plurality of T-lymphocytes expresses a chimeric antigen receptor (CAR); and/or
   the effector cytokine may be Granzyme B, IFN-y, MIP-1a, Performin, TNF-a or TNF-. In certain embodiments, the stimulatory cytokine is GM-CSF, IL-12, IL-15, IL-2, IL-21, IL-5, IL-7, IL-8 and IL-9 (in some embodiments, the chemoattractive cytokine is CCL-I 1, IP-10, MIP-1 or RANTES);

In some embodiments, identifying a subject cell population as efficacious for use in an adoptive cell therapy, the method can further include identifying the subject cell population as safe for use in an adoptive cell therapy, when:
the PSI indicates that at least 50% of the subject cells in the subject cell population are polyfunctional,
the signal intensity of the first signaling molecule indicates that the concentration of the first signaling molecule within the chamber is less than 2 pg/ml,
the signal intensity of the second signaling molecule indicates that the concentration of the second signaling molecule within the chamber is less than 2 pg/ml,
the subject cell population comprises a plurality of T-lymphocytes,
the first signaling molecule comprises a regulatory cytokine or an inflammatory cytokine and
the second signaling molecule comprises a regulatory cytokine or an inflammatory cytokine; and

Accordingly, in some such embodiments:
the regulatory cytokine may be IL-10, IL-13, IL-22, IL-4, TGF-1, sCD137 and sCD40L;
the inflammatory cytokine may be IL-17A, IL-17F, IL-I′ IL-6, MCP-1 and MCP-4;
at least one T-lymphocyte of the plurality of T-lymphocytes may express a chimeric antigen receptor (CAR), and each T-lymphocyte of the plurality of T-lymphocytes may express a chimeric antigen receptor (CAR).

In some embodiments, where identifying a subject cell population as efficacious for use in an adoptive cell therapy, the subject cell population may comprise at least 100 cells, at least 500 cells, at least 1000 cells, or at least 5000 cells.

In identifying a subject cell population as efficacious for use in an adoptive cell therapy, the detecting step, in some embodiments, comprises detecting components of a secretome from each subject cell of the subject cell population, including at least 2 components, at least 10 components, at least 20 components, at least 30 components; at least 50 components, or at least 100 components, of the subject cell population.

In some embodiments, in identifying a subject cell population as efficacious for use in an adoptive cell therapy:
the percentage of polyfunctional cells comprises a first percentage of polyfunctional cells that secrete two or more signaling molecules, a second percentage of polyfunctional cells that secrete three or more signaling molecules, a third percentage of polyfunctional cells that secrete four or more signaling molecules, a fourth percentage of polyfunctional cells that secrete five or more signaling molecules, and a subsequent percentage of polyfunctional cells that secrete increasing numbers of signaling molecules; and/or
the measuring of the signal intensity comprises detecting a fluorescent signal from a complex of an antibody specific for the first or second signaling molecule and the first or second signaling molecule, respectively, and normalizing each fluorescent signal against a reference signal to determine a relative fluorescent unit (RFU) value (in some embodiments, the reference signal is a maximal signal, a minimal signal, or a signal from a component of the secretome with a constant or known concentration, and/or the reference signal is a component of the secretome of a subject cell that is most abundant); and/or
and the method further comprises and one or more of:
   measuring a third or subsequent signaling molecule of the secretome of each polyfunctional cell; and
   determining a relative contribution of the first signaling molecule, the second signaling molecule or the subsequent signaling molecule to a response of the subpopulation of polyfunctional cells to a target cell or to a stimulatory agent, where the relative contribution is the product of an average of a percentage of the PSI of each polyfunctional cell from the first signaling molecule, the second signaling molecule or the subsequent signaling molecule from each polyfunctional cell and a total PSI for the subpopulation of polyfunctional cells.

Support for some of the noted functionality of the present disclosure can be found in PCT/US2017/051223 (published as WO2018/049418) the contents of which is hereby incorporated herein by reference in its entirety.

Accordingly, in some embodiments, target and/or stimulus cells can be cultured overnight in a cell culture flask with serum-free cell culture media. Optionally, culture media can be supplemented with sodium pyruvate, MEM Vitamin Solution, HEPES, Human AB serum, antibiotics, and cytokines. After an overnight recovery, cells may be isolated. Cells may then be cultured at 37° C., 5% CO2 in supplemented cell culture media.

### First Non-Limiting Example: Capture of Cells Using Laser Lysis

Cell Culture. Cells were cultured overnight at 37° C., 5% CO2 in in a cell culture flask with serum-free cell culture media. Culture media was supplemented with sodium pyruvate, MEM Vitamin Solution, HEPES, Human AB serum, antibiotics, and cytokines.

Single-Cell Analysis. Single cells were analyzed to identify its constituent proteins, peptides, polypeptides, nucleic acids, small-molecules, and macromolecules. Prior to performing the single cell assay, a substrate comprising a plurality of isolated chambers of the disclosure was plasma treated to increase hydrophilicity. The substrate was then blocked in BSA/PBS for 30 minutes, and cells were then collected, stained with an anti-human protein antibody and an anti-human protein fluorescent antibody and incubated. Cells were then pelleted via centrifugation and re-suspended in fresh media. Immediately before the assay, the substrate comprising a plurality of isolated chambers was rinsed with media and dried using compressed air. The surface comprising a plurality of capture agents is then positioned on a glass slide and secured into a custom clamping system/device. Cell suspension is pipetted onto the surface comprising a plurality of capture agents, where the surface includes a repeated pattern of antibodies/capture agents, attached thereto. The surface is then contacted to the substrate such that the repeated pattern of antibodies was facing the substrate comprising a plurality of isolated chambers and such that each repeat of the pattern aligned with a chamber of the array. The substrate/cover-surface was then clamped tightly together in the custom clamping system. Individual cells are then isolated into chambers and imaged immediately via fluorescent imaging methods.

Fluorescent Cellular Analysis. The single cell was then analyzed via fluorescence imaging to identify constituent proteins, peptides, polypeptides, nucleic acids, small-molecules, and macromolecules. A fluorescent microscope was used to acquire both bright field and fluorescent images of the substrate comprising a plurality of isolated chambers containing cells. Images of the entire substrate can be taken with a digital camera.

Imaging of Antibody-Patterned Surface. A digital scanner was used to obtain scanned fluorescent images of the surface comprising a plurality of capture agents. Two color channels, 488 (blue, PMT 350, Power 90) and 635 (red, PMT 600, Power 90) can be used to collect fluorescence signals. Obtained images were then be exported and processed to quantify expression levels of components of the cell in each isolated chamber.

Laser Ablation of individual cells. Cells identified to have expression levels of proteins, peptides, polypeptides, nucleic acids, small-molecules, and macromolecules below a pre-determined threshold were targeted for ablation via laser lysis. The entire chamber holding a cell targeted for ablation was illuminated via a laser with sufficient power to result in total cell lysis. The laser need not be focused on the individual cell within the chamber as illumination of the entire chamber is sufficient to result in cellular lysis.

Collection of intact and living cells. Cells identified to have expression levels of proteins, peptides, polypeptides, nucleic acids, small-molecules, and macromolecules above the pre-determined threshold were collected. The releasably coupled substrate comprising chambers and cells and surface comprising a plurality of capture agents were separated. A collection fluid was flowed across the substrate in sufficient volume to dislodge intact cells within the chambers and the fluid containing the homogeneous population of cells was collected for further analysis or processing.

### First Non-Limiting Example: Capture of Cells Via Nano Pipetting

Cell Culture. Cells were cultured overnight at 37° C., 5% CO2 in in a cell culture flask with serum-free cell culture media. Culture media was supplemented with sodium pyruvate, MEM Vitamin Solution, HEPES, Human AB serum, antibiotics, and cytokines.

Single-Cell Analysis. Single cells were analyzed to identify its constituent proteins, peptides, polypeptides, nucleic acids, small-molecules, and macromolecules. Prior to performing the single cell assay, a substrate comprising a plurality of isolated chambers of the disclosure was plasma treated to increase hydrophilicity. The substrate comprising a plurality of isolated chambers was then blocked in BSA/PBS for 30 minutes. Cells were then collected, stained with an anti-human protein antibody and an anti-human protein fluorescent antibody and incubated. Cells were then pelleted via centrifugation and re-suspended in fresh media. Immediately before the assay, the substrate comprising a plurality of isolated chambers can be rinsed with media and dried using compressed air. The surface comprising a plurality of capture agents may then be positioned on a glass slide and secured into a custom clamping system. Cell suspension may then be pipetted onto the surface comprising a plurality of capture agents. A surface with the a repeated pattern of antibodies, also referred to as a surface comprising a plurality of capture agents, attached thereto can be contacted to the substrate comprising a plurality of isolated chambers such that the repeated pattern of antibodies was facing the substrate comprising a plurality of isolated chambers and such that each repeat of the pattern aligned with a chamber of the array. The substrate comprising a plurality of isolated chambers and the surface (with the antibody pattern) enclosing the array can be clamped tightly together in the custom clamping system. Individual cells are then isolated into chambers and imaged immediately via fluorescent imaging methods.

Fluorescent Cellular Analysis. The single cell was then analyzed via fluorescence imaging it identify constituent proteins, peptides, polypeptides, nucleic acids, small-molecules, and macromolecules. A fluorescent microscope was used to acquire both bright field and fluorescent images of the substrate comprising a plurality of isolated chambers containing cells. Images of the entire substrate can be taken with a digital camera.

Imaging of Antibody-Patterned Surface. A digital scanner was used to obtain scanned fluorescent images of the surface comprising a plurality of capture agents. Two color channels, 488 (blue, PMT 350, Power 90) and 635 (red, PMT 600, Power 90) can be used to collect fluorescence signals. Obtained images were then be exported and processed to quantify expression levels of components of the cell in each isolated chamber.

Laser Ablation of individual cells. Cells identified to have expression levels of proteins, peptides, polypeptides, nucleic acids, small-molecules, and macromolecules below a pre-determined threshold were targeted for ablation via laser lysis. The entire chamber holding a cell targeted for ablation was illuminated via a laser with sufficient power to result in total cell lysis. The laser need not be focused on the individual cell within the chamber as illumination of the entire chamber is sufficient to result in cellular lysis.

Collection of intact and living cells. Cells identified to have expression levels of proteins, peptides, polypeptides, nucleic acids, small-molecules, and macromolecules above the pre-determined threshold were collected. The releasably coupled substrate comprising chambers and cells and surface comprising a plurality of capture agents were separated. Individual cells can then be collected from their respective chambers via nanopipette and either stored individually or pooled to form a homogeneous population of cells.

### Definitions

Unless otherwise defined, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The practice of at least some of the embodiments of the present disclosure can employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The term “antibody” (Ab) as used herein includes monoclonal antibodies, polyclonal antibodies, multispecific antibodies (e.g., bispecific antibodies), and antibody fragments, as long as they exhibit the desired biological activity. The term “immunoglobulin” (lg) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Capture agents of the disclosure may comprise one or more monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

Monoclonal antibodies contemplated herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Capture agents of the disclosure may comprise humanized antibodies. A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed by substituting import hypervariable region sequences for the corresponding sequences of a human antibody. Accordingly, such “humanized” antibodies are chimeric antibodies wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

Capture agents of the disclosure may comprise intact antibodies. An “intact” antibody is one that comprises an antigen-binding site as well as a CL and at least heavy chain constant domains, CH 1, CH 2 and CH 3. The constant domains may be native sequence constant domains (e.g., human native sequence constant domains) or amino acid sequence variant thereof. Preferably, the intact antibody has one or more effector functions.

Capture agents of the disclosure may comprise an antibody fragment. An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

Capture agents of the disclosure may comprise a functional fragment or an analog of an antibody. The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fci: RI.

Papain digestion of antibodies produces two identical antigen-binding fragments, called “Fab” fragments, and a residual “Fe” fragment, a designation reflecting the ability to crystallize readily. The Fab fragment consists of an entire L chain along with the variable region domain of the H chain (VH), and the first constant domain of one heavy chain (CH 1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “Fe” fragment comprises the carboxy-terminal portions of both H chains held together by disulfides. The effector functions of antibodies are determined by sequences in the Fe region, which region is also the part recognized by Fe receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the Hand L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Capture agents of the disclosure may comprise single-chain antibodies (also referred to as scFv). “Single-chain Fv” also abbreviated as “sFv” or “scFv” are antibody fragments that comprise the VH and VL antibody domains connected into a single polypeptide chain.

Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

Capture agents of the disclosure may comprise diabodies. The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

Capture agents of the disclosure may comprise bispecific antibodies. In certain embodiments, antibodies are bispecific or multi-specific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Methods for making bispecific antibodies are known in the art. Traditional production of full length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable domains with the desired binding specificities (antibody-antigen combining sites) are fused to immunoglobulin constant domain sequences. Preferably, the fusion is with an lg heavy chain constant domain, comprising at least part of the hinge, CH2, and CH3 regions. It is preferred to have the first heavy-chain constant region (CHI) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

As used herein, an antibody is said to be “immunospecific,” “specific for” or to “specifically bind” an antigen if it reacts at a detectable level with the antigen, preferably with an affinity constant, Ka, of greater than or equal to about 104 M·1, or greater than or equal to about 105 M·1, greater than or equal to about 106 M·1, greater than or equal to about 107 M·1, or greater than or equal to 108 M-1. Affinity of an antibody for its cognate antigen is also commonly expressed as a dissociation constant KD, and in certain embodiments, an antibody specifically binds to a component of a secretome if it binds with a KD of less than or equal to 10-4 M, less than or equal to about 10-5 M, less than or equal to about 10-6 M, less than or equal to 10-7 M, or less than or equal to 10-8 M. Affinities of antibodies can be readily determined using conventional techniques, for example, those described by Scatchard et al. (Ann. N.Y. Acad. Sci. USA 51:660 (1949)).

Subject and target cells of the disclosure may be isolated, derived, or prepared from any species, including any mammal. A “mammal” for purposes of treating n infection, refers to any mammal, including humans, domestic and farm animals, and zoo, sports, or pet animals, such as dogs, cats, cattle, horses, sheep, pigs, goats, rabbits, etc. Preferably, the mammal is human.

Subject cells of the disclosure may be used in a cellular therapy for the treatment of a disease or disorder. “Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal may be successfully “treated” when, after receiving a cellular therapy with a subject cell of the disclosure, the patient shows observable and/or measurable reduction in or absence of one or more of the following: reduction in one or more of the symptoms associated with disease or disorder; reduced morbidity and mortality, and improvement in quality of life issues. The above parameters for assessing successful treatment and improvement in the disease are readily measurable by routine procedures familiar to a physician. Methods of the disclosure may be used to determine the safety and/or efficacy of a cellular therapy before, during or after initiation of treatment of the subject.

Capture agents of the disclosure may be labeled to render them detectable using one or more means. “Label” as used herein refers to a detectable compound or composition that is conjugated directly or indirectly to the capture agent (e.g. an antibody) so as to generate a “labeled” capture agent (e.g. an antibody). The label may be detectable by itself (e.g., a fluorescent label) or, in the case of an enzymatic label, may catalyze chemical alteration of a substrate compound or composition that is detectable.

Capture agents of the disclosure may selectively or specifically identify, capture, and/or quantify one or more small molecules in a secretome. A “small molecule” is defined herein to have a molecular weight below about 500 Daltons.

Capture agents of the disclosure may include nucleic acids or labeled nucleic acids. The terms “nucleic acid” and “polynucleotide” are used interchangeably herein to refer to single- or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences according to some embodiments of the disclosure, and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of at least some embodiments of the present disclosure, and evaluating one or more biological activities of the polypeptide as described herein and/or using any of a number of techniques well known in the art. Modifications may be made in the structure of the polynucleotides and polypeptides of the disclosure and still obtain a functional molecule that encodes a variant or derivative polypeptide with desirable characteristics. When it is desired to alter the amino acid sequence of a polypeptide to create an equivalent, or even an improved, variant or portion of a polypeptide of at least some embodiments of the present disclosure, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences that encode said peptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5).

Certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. The substitution of like amino acids can be made effectively on the basis of hydrophilicity. The greatest local average hydrophilicity of a protein, as governed by the hydrophilicity of its adjacent amino acids, correlates with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (O); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D J. (1983) Proc. Natl. Acad., Sci. USA 80:726-730.

Alternatively, optimal alignment of sequences for comparison may be conducted by the local identity algorithm of Smith and Waterman (1981) Add APL. Math 2:482, by the identity alignment algorithm of Needleman and Wunsch (1970) J Mal. Biol. 48:443, by the search for similarity methods of Pearson and Lipman (1988) Proc. Natl. Acad Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J Mal. Biol. 215:403-410, respectively. BLAST and BLAST 2.0 can be used, for example with the parameters described herein, to determine percent sequence identity for the polynucleotides and polypeptides according to some embodiments of the disclosure. Software for performing BLAST analyses is publicly available through the National Center for Biotechnology Information.

“Homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least 70%, at least 75%, at least 80%, at least 90%, at least 95%, at least 98%, or at least 99% polynucleotide or polypeptide homology with a polynucleotide or polypeptide described herein.

### ADDITIONAL REMARKS

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims, equivalents thereto, and any claims supported by the present disclosure, inventive embodiments may be practiced otherwise than as specifically described and claimed. Inventive embodiments of the present disclosure are directed to each individual feature, system, article, material, kit, method, and step, described herein. In addition, any combination of two or more such features, systems, articles, materials, kits, methods, and steps, if such features, systems, articles, materials, kits, methods, and steps, are not mutually inconsistent, is included within the inventive scope of the present disclosure. Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is currently claimed:

1. A selective cell collection and/or sorting method for at least one of selectively collecting and sorting cells comprising:
loading or otherwise placing into each of a plurality of isolated chambers of a substrate, a cell and a volume of fluid, wherein:
the substrate including a first surface that is releasably coupled to a transparent cover having a second surface, forming an assembly,
the second surface having a plurality of capture agents, and
the volume of fluid is in fluid communication with the second surface;
maintaining each cell under one or more conditions sufficient to permit:
the production of one or more cellular components by each cell, and
the one or more cellular components configured to bind with at least one of the capture agents of the surface so as to form at least one capture agent cellular component complex;
for each of the cells, detecting the at least one capture agent cellular component complex;
identifying at least one cell for at least one of collection and removal; and
collecting the at least one cell.

2. The method of claim 1, further comprising ablating the cell identified for removal.

3. The method of claim 2, wherein ablating comprises contacting a respective isolated chamber comprising the cell for removal with a laser.

4. The method of claim 3, wherein the laser is configured to lyse the cell.

5. The method of any of claims 1-4, wherein a plurality of cells comprises a heterogeneous population of cells.

6. The method of claim 5, wherein the heterogeneous cell population is a functionally heterogeneous cell population.

7. The method of claim 6, wherein:
the functionally heterogeneous cell population comprises at least two cells that produce a secretome in response to a stimulus,
a first cell of the at least two cells produces a first secretome,
a second cell of the at least two cells produces a second secretome, and
wherein the first secretome and the second secretome are not identical.

8. The method of claim 6, wherein the functionally heterogeneous cell population comprises one or more immune cells.

9. The method of claim 8, wherein the one or more immune cells comprise a T-lymphocyte, a B-lymphocyte, a natural killer (NK) cell, a macrophage, a neutrophil, a mast cell, an eosinophil, or a basophil.

10. The method of claim 9, wherein the T-lymphocyte comprises a native T-lymphocyte, an activated T-lymphocyte, an effector T-lymphocyte, a helper T-lymphocyte, a cytotoxic T-lymphocyte, a gamma-delta T-lymphocyte, a regulatory T-lymphocyte, a memory T-lymphocyte, or a memory stem T-lymphocyte.

11. The method of claim 9 or 10, wherein the T-lymphocyte expresses at least one of:
a non-naturally occurring antigen receptor; and
a Chimeric Antigen Receptor (CAR).

12. The method of claim 9, wherein the B-lymphocyte comprises a plasmablast, a plasma cell, a memory B-lymphocyte, a regulatory B cell, a follicular B cell, or a marginal zone B cell.

13. The method of claim 6, wherein the functionally heterogeneous cell population comprises:
one or more neuronal cells, the one or more neuronal cells comprising a neuron, a glial cell, an astrocyte, a satellite cell, or an enteric glial cell;
one or more endocrine cells, the one or more endocrine cells being isolated or derived from a pineal gland, a pituitary gland, a pancreas, an ovary, a testicle, a thyroid gland, a parathyroid gland, a hypothalamus, or an adrenal gland; and
one or more exocrine cells, the one or more exocrine cells are isolated or derived from a salivary gland, a sweat gland or a component of the gastrointestinal tract.

14. The method of any one of claims 1-13, wherein the one or more conditions comprise contacting each cell with a stimulus.

15. The method of claim 14, wherein the stimulus is selected from the group consisting of:
an ion, a small molecule, a nucleic acid sequence, a peptide, a polypeptide, a protein, a ligand, a receptor, an antigen, a cell or organelle membrane patch, a cell or organelle membrane, a cell, or any combination thereof;

16. The method of claim 14, wherein the stimulus:
is naturally-occurring or not naturally-occurring;
is operably-linked to an interior surface of each respective chamber;
is the volume of fluid in the chamber;
and/or
recapitulates the effect of the subject cell contacting a target cell.

17. The method of claim 16, wherein the target cell is a deleterious cell; and/or
the target cell is selected from the group consisting of: a proliferating cell, a cancer cell, an infected cell, a foreign cell, or an immune cell.

18. The method of claim 17, wherein the foreign cell is a bacteria, a yeast, or a microbe,

19. The method of claim 16, wherein the target cell is a healthy cell.

20. The method of claim 19, wherein the healthy cell is a B-lymphocyte.

21. The method of any one of claims 1-20, wherein the one or more conditions comprise maintaining each of the plurality of cells in a cell media that maintains the viability of each cell.

22. The method of any of claims 1-21, wherein the volume of fluid in each of the plurality of isolated chambers includes a cell media that maintains the viability of each cell.

23. The method of any one of claims 1-22, wherein:
the capture agents are arranged on the surface in a repeated pattern, and
the substrate and the surface are releasably coupled such that at least one repeat of the repeated pattern of capture agents is enclosed in each chamber of the plurality of chambers.

24. The method of any one of claims 1-23, wherein:
the one or more cellular components comprise a secretome, and
the secretome comprises one or more distinct peptides, polypeptides, or proteins that indicates diminished or decreasing cell function or cell viability.

25. The method of any one of claims 1-24, wherein:
the one or more cellular components comprise a secretome, and
the secretome comprises one or more distinct peptides, polypeptides, or proteins that indicates augmented or increasing inflammation, or indicates increased cell activity or cellular stimulation.

26. The method of any one of claims 1-25, wherein the identifying step comprises determining a Polyfunctional Strength Index (PSI) for each of the at least two cells.

27. The method of claim 26, wherein the PSI is the product of a percentage of polyfunctional subject cells within the heterogeneous cell population and an average signal intensity of two or more cytokines.

28. The method of claim 27, wherein the polyfunctional subject cells, at a single cell level, secrete at least two cytokines.

29. The method of claim 28, wherein the at least two cytokines produced by each of the polyfunctional subject cells and the two or more cytokines of the average signal intensity comprise the same cytokines.

30. The method of claim 28, wherein the at least two cytokines produced by each of the polyfunctional subject cells and the two or more cytokines of the average signal intensity consist of the same cytokines.

31. The method of any one of claims 26-30, wherein an increase in the PSI indicates an increase in the potency of the polyfunctional subject cells.

32. The method of any of claims 3-31, wherein during the ablation step, the laser does not directly contact the cell identified for removal.

33. The method of any of claims claim 3-32, wherein the laser is selected from the group consisting of: a diode, helium-neon, argon, xenon, nitrogen, carbon dioxide, carbon monoxide, hydrogen fluoride, deuterium fluoride, helium-cadmium, helium-mercury, helium-selenium, helium-silver, or neon-copper laser.

34. The method of any of claims 1-33, wherein collecting comprises separating the surface from the substrate, and collecting the cell identified for collection.

35. The method of claim 34, wherein the separated substrate and cover of the assembly form an inlet and an outlet and a flow channel therebetween.

36. The method of claim 34 or 35, wherein collection comprises at least one of nano-pipetting each cell identified for collection out of each respective chamber, and flowing a collection fluid.

37. The method of claim 36, wherein flowing the collection fluid includes from the inlet to the outlet, the flow of the fluid configured to retain collect cells identified for collection.

38. The method of claim 36, wherein flowing a fluid also includes mechanically vibrating the substrate so as move cells identified for collection into the collection fluid flow.

39. The method of claim 34, wherein the collection fluid containing the collected cells comprise a composition.

40. The method of claim 39, wherein a composition comprises the collected cell.

41. The method of any of claims 39-40, further comprising purifying the composition to remove one or more of a stimulus, a reagent, a cell media, one or more cellular components, one or more components of a secretome, a secreted protein, an intracellular component, cell debris or any combination thereof.

42. The method of any of claims 39-41, wherein the composition further comprises a media to maintain viability or polyfunctionality of the cell.

43. The method of any of claims 1-42, wherein the method further comprises contacting the collected cell with an expansion composition.

44. The method of any one of claims 1-43, further comprising analyzing the collected cell or a component thereof.

45. The method of claim 44, wherein the analyzing step comprises one or more of DNA sequencing, RNA sequencing, genomics analysis, and proteomics analysis.

46. The method of any of claims 1-45, wherein detecting comprises:
exposing the cover of the assembly to a light source configured to fluoresce formed complexes, and
imaging the cover for fluorescing complexes.

47. The method of claim 46, wherein imaging the cover includes taking a plurality of images of overlapping portions of both the first and second surfaces.

48. The method of claim 46 or 47, wherein the second surface is configured with a plurality of fiducial lines and the substrate is configured with a plurality of organized fiducial marks.

49. The method of claim 48, identifying comprises using the fluorescence and lines and marks of the images to locate one or more of the chambers to which the fluorescence corresponds.

50. The method of claim 49, wherein the images are first assembled together.

51. The method of any of claims 48-50, wherein fiducial lines are arranged on the second surface in a repeated, pre-defined pattern.

52. The method of any of claims 48-51, wherein each fiducial line includes a unique color.

53. The method of any of claims 48-52, wherein the fiducial marks are arranged on the first surface in-between columns of the chambers.

54. The method of claim 53, wherein the marks are configured with two or more different length of with equal spacing between.

55. The method of claim 54, wherein chambers in between the spacing is labeled.

56. A selective cell sorting and/or collection system comprising:
a dock suitable for receiving a device comprising a substrate and a transparent cover including a second surface, wherein:
the substrate includes a plurality of isolated chambers and a first surface; and
the second surface includes a plurality of capture agents, and
coupling of the substrate and the cover encloses each chamber of the plurality of chambers;
the dock comprises a clamp configured to releasably couple the substrate and the cover of the device;
a first optics system comprising a first objective and configured to:
move along the X, Y, and Z axes, and
produces at least one of a visible and a fluorescent light;
optionally a second optics system comprising a second objective and configured to:
moves along the X, Y, and Z axes, and
produce an ablating laser beam;
a first detector optionally corresponding to the first optics system; and
a processor configured to at least one of process information of the system and control the system or one or more components thereof;
wherein the first optics system may be positioned between the dock and the first detector.

54. The system of claim 46, wherein the laser is selected from the group consisting of: a diode helium-neon, argon, xenon, nitrogen, carbon dioxide, carbon monoxide, hydrogen fluoride, deuterium fluoride, helium-cadmium, helium-mercury, helium-selenium, helium-silver, or neon-copper laser.

58. The system of claim 53 or 54, wherein the detector comprises a digital camera.

59. The system of claim 58, wherein:
the first optics system is configured to expose the cover of the assembly to the fluorescent light so as to fluoresce formed complexes, and
the detector images the cover for the fluorescing complexes.

60. The system of any of claims 53-59, wherein the detector obtains a plurality of images of overlapping portions of both the first and second surfaces.

61. The system of claim 59 or 60, wherein the second surface is configured with a plurality of fiducial lines and the substrate is configured with a plurality of organized fiducial marks.

61. The system of claim 48, wherein the processor is configured to process image information so as to identify one or more chambers upon which fluorescence is captured in the images.

62. The system of claim 61, wherein the processor uses the fluorescence and lines and marks of the images to locate the one or more of the chambers.

63. The system of claim 49, wherein the processor is configured to assemble adjacent images together.

64. The system of any of claims 61-63, wherein fiducial lines are arranged on the second surface in a repeated, pre-defined pattern.

65. The system of any of claims 61-64, wherein each fiducial line includes a unique color.

66. The system of any of claims 61-65, wherein the fiducial marks are arranged on the first surface in-between columns of the chambers.

67. The system of claim 66, wherein the marks are configured with two or more different length of with equal spacing between.

68. The system of claim 67, wherein chambers in between the spacing is labeled.
